(19) United States
(12) Patent Application Publication
(10) Pub. No.: US 2026/0334848 A1
(43) Pub. Date: Oct. 29, 2026

(54) COGNITIVE IMPAIRMENT DETECTION SYSTEM AND THE DETECTION METHOD THEREOF

(71) Applicant: NATIONAL YANG MING CHIAO TUNG UNIVERSITY, Hsinchu City (TW)

(72) Inventors: Li-Chun Wang, Hsinchu City (TW); Wei-Zheng Chen, Hsinchu City (TW); Chieh-Ming Yang, Shoufeng Township (TW); Kuan-Chih Huang, Kaohsiung City (TW)

(21) Appl. No.: 19/319,262

(22) Filed: Sep. 4, 2025

(30) Foreign Application Priority Data
Apr. 28, 2025 (TW) 114115969

(51) Int. Cl. A61B 5/372; A61B 5/00

(52) U.S. Cl. CPC A61B 5/372; A61B 5/4088; A61B 5/7264

(57) ABSTRACT

A cognitive impairment detection system comprising: a data collection module having multiple collection electrodes, each of collection electrode collects an original electroencephalogram (original EEG) from a corresponding encephalic regions of a participant; a data processing module receiving the original electroencephalogram (original EEG) and performing a data processing procedure on the original EEG to form a processed electroencephalogram (processed EEG); and a feature extraction module receiving the processed EEG and classifying the participant; wherein the feature extraction module comprising: a power spectral density module (PSD module) based on a power of the processed EEG in a first frequency band classifies whether the participant is a dementia patient by using a first supervised learning method; and a coherence module selecting the processed EEG from different encephalic region for coherence analysis, using a second supervised learning method to classify the non-dementia participants into healthy controls (HC) and mild cognitive impairment (MCI).

### CROSS-REFERENCE TO RELATED APPLICATIONS

This application claims the benefit of TW 114115969, filed Apr. 28, 2025, which is incorporated in its entirety by reference herein.

### FIELD OF THE INVENTION

The present invention relates to a cognitive impairment detection system, particularly relates to a cognitive impairment detection system based on electroencephalogram (EEG) analysis and the detection method thereof.

### BACKGROUND OF THE INVENTION

With the intensifying trend of global population aging, healthcare systems face unprecedented challenges, particularly in the management and care of chronic diseases such as dementia. According to a report by Alzheimer's Disease International (ADI), more than 50 million people worldwide suffered from dementia in 2019, and this number is expected to increase to 152 million by 2050. The annual economic burden associated with dementia is as high as one trillion dollars, placing immense pressure on the global economy.

As the physiological systems of the elderly gradually weaken with age, this not only brings numerous physical challenges but also significantly increases the demand and cost of healthcare. The growing elderly population means a continuous rise in the need for medical resources, especially in long-term care and specialized treatments. According to a report by Organisation for Economic Co-operation and Development (OECD), global healthcare expenditure will increase significantly in the coming decades due to the population aging, placing a greater burden on the healthcare systems of various countries.

Mild Cognitive Impairment (MCI) is a common intermediate stage between normal aging and dementia. Over time, MCI may progress into more severe neurodegenerative diseases. Therefore, early diagnosis of MCI and taking preventive precautions are important for controlling healthcare costs and improving the quality of life for patients. Early diagnosis and intervention can not only delay the progression of the disease but also reduce the burden on patients and their families. These challenges have prompted researchers and healthcare professionals to continuously seek innovative technologies and methods to improve the early diagnosis and treatment of dementia and MCI.

The integration of advanced healthcare technologies and data analysis methods to more accurately identify these diseases and provide better care and support for patients has become an extremely important issue today's landscape.

### SUMMARY OF THE INVENTION

The primary objective of the present invention is to obtain electroencephalogram (EEG) from participants, and subsequently classify and detect the individuals with mild cognitive impairment (MCI), so as to achieve the effect of early treatment.

According to the above objective, the present invention provides a cognitive impairment detection system comprising: a data collection module having the plurality of collection electrodes, each of the plurality of collection electrode collects an original electroencephalogram (original EEG) from the corresponding encephalic regions of the participant; a data processing module receiving the original EEG and performing a data processing procedure on the original EEG to form a processed electroencephalogram (processed EEG), such as signal sampling, signal filtering, and artifact removal; and a feature extraction module receiving the processed EEG and classifying the participant. Wherein the feature extraction module comprising: a power spectral density module (PSD module) based on a power of the processed EEG in a first frequency band classifies whether the participant is a dementia patient by using a first supervised learning method; and a coherence module selecting the processed EEG from different encephalic region for coherence analysis, using a second supervised learning method to classify the non-dementia participants into healthy controls (HC) and mild cognitive impairment (MCI).

According to the above objective, the present invention further provides a cognitive impairment detection method, comprising: collecting an original electroencephalogram (original EEG) from the corresponding encephalic regions of the participant; processing the original EEG to form a processed electroencephalogram (processed EEG); performing feature extraction step to classify the participant, wherein the feature extraction step comprising: extracting a power in the first frequency band based on the processed EEG and using a first supervised learning method to determine whether a participant is a dementia patient; and performing a second supervised learning method based on the coherence analysis in the processed EEG to determine whether the non-dementia participants is a healthy controls (HC) or a mild cognitive impairment (MCI).

### BRIEF DESCRIPTION OF THE DRAWINGS

FIG. 1 is a block diagram illustrating an embodiment of a cognitive impairment detection system according to the technology disclosed present invention.

FIG. 2 is an analysis results of the power spectral density analysis illustrating an embodiment of a cognitive impairment detection system according to the technology disclosed present invention.

FIG. 3 is an analysis results of the coherence analysis illustrating an embodiment of a cognitive impairment detection system according to the technology disclosed present invention.

FIG. 4 is a flow chart illustrating an embodiment of a cognitive impairment detection method according to the technology disclosed present invention.

### DETAILED DESCRIPTION OF THE PREFERRED EMBODIMENT

Detailed descriptions of embodiments of the present invention are given below; however, the scope of the present invention is not limited by the embodiments described herein. The figures herein are all schematic representations and are mainly used to show the connection between units. The embodiments are described in detail below with reference to the accompany figures.

The data of the electroencephalogram (EEG) is collected through 21 electrodes deployed on the scalp. According to the International recognized 10-20 system nomenclature, the electrode positions include: left frontopolar (Fp1), right frontopolar (Fp2), left frontal (F3), right frontal (F4), left central (C3), right central (C4), left parietal (P3), right parietal (P4), left occipital (O1), right occipital (O2), left frontotemporal (F7), right frontotemporal (F8), left temporal (T3), right temporal (T4), left posterior temporal (T5), right posterior temporal (T6), left mastoid (A1), right mastoid (A2), midline frontal (Fz), central midline (Cz), midline parietal (Pz). Among them, A1 and A2 are used as reference electrodes for EEG signals.

Power spectral density (PSD) is a technique used to quantify the energy distribution of EEG signals in the frequency domain. By performing time-frequency transformation on EEG signals, including Short-time Fast Fourier Transform (STFFT) or Wavelet Transform (WT), the energy distribution across different frequency bands can be obtained, thereby enabling the characterization of frequency components associated with brain activity. The present invention segments the original EEG signals and performs STFFT or WT calculation on each segment, then averages the PSD results of multiple segments, as shown in the equation,

$$P S D ( f ) = \frac{1}{N \cdot Δ t} {\sum }_{k = 1}^{K} {\vert {F}_{k} ( f ) \vert}^{2} .$$

PSD(f) represents the estimate of power spectrum density at frequency f. N represents the number of data points in each segment, which is 1000 in the present invention, corresponding to 2 seconds of data. Δt represents the time interval between each data point, Δt=1/500=0.002 seconds. K represents the total number segments of divided signal, where 10 seconds of data is divided into 9 segments, with 50% overlap between each segment. Fk(f) represents the value of STFFT or WT result in the k-th segment of data at frequency f.

Coherence is a statistic used to quantify the degree of synchronization between two signals at a specific frequency. The value of coherence is between 0 and 1, where 0 indicates complete lack of synchronization and 1 indicates complete synchronization. In the present invention, the coherence between 19 EEG channels is calculated, with a total of C₂¹⁹=171 pairs of channels. The 10-second data is divided into 2-second segments (1000 data points) at the pre-resting stage, and STFFT or WT is performed. In order to ensure the continuity and stability of time, there is a 50% overlap between each segment. The coherence calculation is performed on each segment of each pair of channels, and then the coherence calculation results of multiple segments are averaged. The coherence calculation is shown as the formula

$${C}_{x y} ( f ) = \frac{{\vert {P}_{xy} ( f ) \vert}^{2}}{{P}_{x x} ( f ) \cdot {P}_{yy} f} .$$

Cxy(f) is the coherence between signal x(t) and signal y(t) at frequency f. Pxy(f) is respectively the cross-power spectrum of signal x(t) and signal y(t) at frequency f, as shown in the formula

$${P}_{xy} ( f ) = \frac{1}{K} {\sum }_{k = 1}^{K} {F}_{k}^{( x )} ( f ) \cdot {{F}_{k}^{( y )} ( f )}^{*} ,$$

wherein

$${F}_{k}^{( x )} ( f ) and {F}_{k}^{( y )} ( f )$$

are the time-frequency conversion results of signal x(t) and signal y(t) in the k-th segment. Pxx(f) and Pyy(f) are the power spectral densities (PSDs) of signal x(t) and signal y(t) at frequency f. Coherence reflects the synchronization of two signals at a certain frequency, and the degree of synchronization is quantified by comparing the cross-power spectrum with the power spectrum of the two signals.

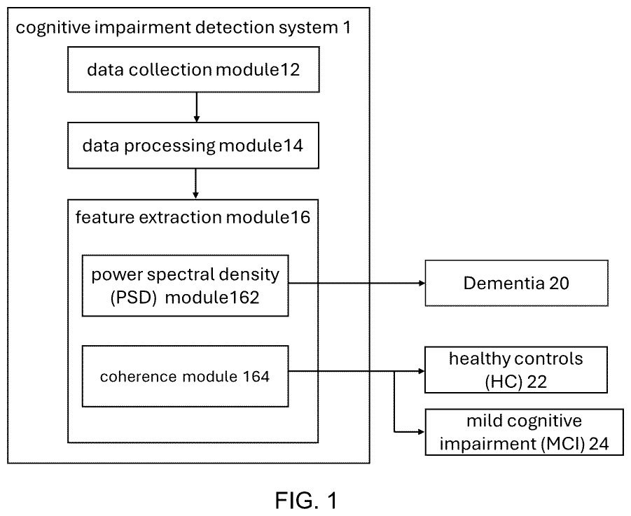

First, please refer to FIG. 1. FIG. 1 represents a cognitive impairment detection system 1 according to the technology disclosed in the present invention, comprises: a data collection module 12, a data processing module 14, and a feature extraction module 16. The feature extraction module 16 comprises: a power spectral density (PSD) module 162 and a coherence module 164. The data collection module 12 has the plurality of collection electrodes, each of the plurality of collection electrode collects original electroencephalogram (original EEG) from corresponding encephalic region of participant. The data processing module 14 receives the original EEG and performs data processing procedure on the original EEG to form a processed electroencephalogram (processed EEG), such as signal sampling, signal filtering, and artifact removal. The feature extraction module 16 receives the processed EEG and classifying the participant. The PSD module 162 based on power of processed EEG in first frequency band classifies whether participant is dementia patient by using first supervised learning method. Coherence module 164 selects processed EEG from different encephalic regions for coherence analysis and uses second supervised learning method to classify non-dementia participants into healthy controls (HC) and mild cognitive impairment (MCI).

In one embodiment of the present invention, the collection electrodes of data collection module 12 collects original EEG when participant is in resting state. EEG collected in resting state is an EEG collecting participant's natural neural activity without external interference. Compared with the EEG obtained in other states, the EEG collected in resting state can effectively classify the participants into dementia patients, healthy controls, and mild cognitive impairment.

In one embodiment of the present invention, data processing module 14 performs a data processing procedure which performs artifact removal from original EEG to obtain processed EEG. This procedure can avoid excessive noise interference during classification and be capable of preliminarily selecting more suitable data for subsequent feature extraction. The data processing procedure may use the following processing methods, such as baseline correction, band-pass filter, independent component analysis, artifact subspace reconstruction, and t-test.

In one embodiment of the present invention, the feature extraction module 16 comprises PSD module 162 and coherence module 164, which are used to receive the processed EEG and classify the participant. The PSD module 162 based on a power of the processed EEG in a first frequency band classifies whether the participant is dementia patient by using first supervised learning method. In PSD module, the first frequency band is in a relatively low frequency range, such as 0.5 to 20 hertz (Hz). The preferred range of the first frequency band is 0.5 to 8 Hz, 1 to 6 Hz or 0.5 to 5 Hz. The first supervised learning method is support vector machine (SVM), K-nearest neighbors (KNN), decision tree, random forest, logistic regression, Extreme gradient boosting (XGBoost), neural networks, or Naive bayes classifier. The preferred first supervised learning method is support vector machine. Coherence module 164 selects the processed EEG from different encephalic region for coherence analysis, and uses second supervised learning method to classify non-dementia participants into healthy controls (HC) and mild cognitive impairment (MCI). The second supervised learning method is support vector machine, K-nearest neighbors, decision tree, random forest, logistic regression, Extreme gradient boosting, neural networks, or Naive bayes classifier. The preferred second supervised learning method is support vector machine.

In one embodiment of the present invention, the collection electrodes of data collection module 12 collects original EEG in resting state. The data processing module 14 uses baseline correction, band-pass filter, independent component analysis to perform artifact removal of original EEG, and uses statistical tests, such as t-test, to preliminarily select frequency bands with significant differences. The PSD module 162 of feature extraction module 16 performs power spectrum density analysis based on at least two channels from the frontal lobe and the temporal lobe of encephalic regions. It should be noted that in the present invention, the aforesaid encephalic regions can preferably be left frontopolar (Fp1), left frontal (F3), right frontal (F4), right posterior temporal (T6), midline parietal (Pz), or midline central (Cz). The PSD module 162 preferably uses at least five channels for power spectrum density analysis. Then, a low frequency band is selected from PSD analysis results, such as 0.5 to 80 Hz, 1 to 6 Hz, 0.5 to 8 Hz or 0.5 to 5 Hz. Subsequently the support vector machine method is used to determine whether the participant is a dementia patient.

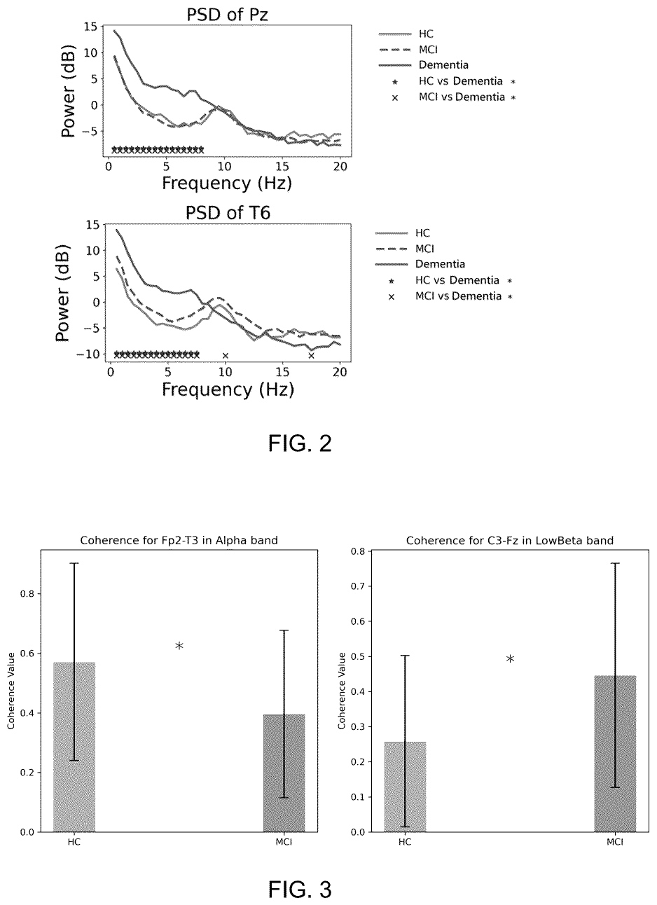

Next, please refer to FIG. 2. FIG. 2 shows a cognitive impairment detection system 1 disclosed in the present invention. The results of the power spectral density analysis are performed by PSD module 162 of feature extraction module 16 are illustrated. In encephalic regions such as midline parietal (Pz) and right posterior temporal (T6), there is significant difference in low frequency bands between healthy control and dementia patients or between mild cognitive impairment and dementia patients, particularly in 1 to 6 Hz.

The coherence module 164 selects a combination of at least two channels group from frontal lobe and the other encephalic region to perform coherence analysis on the processed EEG. It should be noted that in the present invention combination of channels group is right frontopolar (Fp2) and left temporal (T3), midline frontal (Fz) and left central (C3), right frontal (F4) and left central (C3) for coherence analysis. Coherence module 164 preferably uses at least five channels for coherence analysis. Subsequently the support vector machine method is used to classify non-dementia participants into healthy controls (HC) and mild cognitive impairment (MCI).

Please refer to FIG. 3. FIG. 3 shows a cognitive impairment detection system 1 disclosed in the present invention. The results of the coherence analysis are performed by coherence module 164 of feature extraction module 16 are illustrated. The combination of channels group between right frontopolar (Fp2) and left temporal (T3) in Alpha band, which frequency ranges from 8 Hz to 12 Hz, and the combination of channels group between midline frontal (Fz) and left central (C3) in Low Beta band, which frequency ranges from 12 Hz to 15 Hz are particularly significant between healthy controls (HC) and mild cognitive impairment (MCI).

In the present invention an embodiment of cognitive impairment detection system is classified by the aforesaid embodiment. Please refer to Table 1. The results indicate that PSD analysis in low frequency band can significantly distinguish dementia patients from other categories. Moreover, coherence analysis shows significant difference between healthy control (HC) and mild cognitive impairment (MCI), particularly in the weakening of coherence between specific encephalic regions. Statistical analysis confirmed after the above embodiment was classified, that the sensitivity is 0.7897, the specificity is 0.8884, the accuracy is 0.8111, and balanced accuracy is 0.8390. These results show that the combination of the PSD module and the coherence module can improve the classification accuracy of the cognitive impairment detection system, which is helpful for improving clinical diagnosis.

TABLE 1

| | | True category | | |
|---|---|---|---|---|
| Classification result | | HC | MCI | dementia |
| Classification | HC | 15 | 4 | 1 |
| category | MCI | 3 | 42 | 4 |

| | | |
|---|---|---|
| dementia | 5 | 16 |

It should be noted that the use of the PSD module alone for classification can only determine whether the participant is dementia patient but cannot distinguish the mild cognitive impairment. The accuracy of classification when using the coherence module alone is only approximately 50%. In the present invention, the feature extraction module 16 uses the PSD module 162 for preliminary classification at first, followed by the coherence module 164 for further refinement, and achieves an accuracy exceeding 80%.

Furthermore, the cognitive impairment detection system of the present invention provides an important reference for the early detection of mild cognitive impairment and has the potential applications in personalized medicine. In the future, the system can be combined with non-invasive brain stimulation technology, such as transcranial direct current stimulation (tDCS), to optimize treatment parameters and achieve precise localization, thereby enhancing therapeutic efficacy and reducing patient burden.

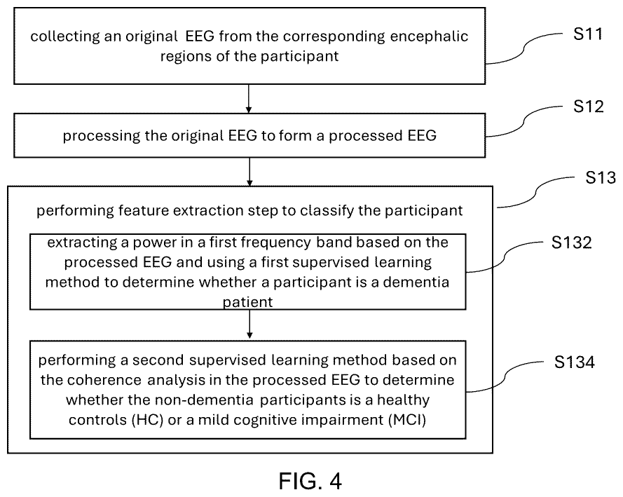

In addition, please refer to FIG. 4. FIG. 4 is a flow chart of the steps of cognitive impairment detection method. In step S11, it collects an original EEG from the corresponding encephalic regions of the participant. In step S12, it processes the original EEG to form a processed EEG. In step S13, it performs feature extraction step to classify the participant, wherein the step S13 comprises step S132 and step S134. In step S132, the power in the first frequency band is extracted based on the processed EEG and using the first supervised learning method to determine whether the participant is a dementia patient. In step S134, based on the coherence analysis in the processed EEG, a second supervised learning method is used to determine whether the non-dementia participants is a healthy control group or a mild cognitive impairment patient.

The cognitive impairment detection method can refer to the cognitive impairment detection system to adjust the corresponding parameters, states, data processing procedures and frequency band range. Simultaneously, the criteria of first supervised learning method and second supervised learning method can be adjusted according to the needs by the cognitive impairment detection system.

The foregoing descriptions are only preferred embodiments of the present invention and are not used to limit the scope of the present invention. Meanwhile, any person with ordinary knowledge in the art can easily understand and implement it. Therefore, any equivalent variation or modification without departing from the spirit of the present invention disclosed herein is to be included within the scope of the present invention.

What is claimed is:

1. A cognitive impairment detection system, comprising:
a data collection module having a plurality of collection electrodes, each of the plurality of collection electrode collects an original electroencephalogram (original EEG) from a corresponding encephalic region of a participant;
a data processing module receiving the original EEG and performing an data processing procedure on the original EEG to form a processed electroencephalogram (processed EEG); and
a feature extraction module receiving the processed EEG and classifying the participant, wherein the feature extraction module comprising:
a power spectral density module (PSD module) based on a power of the processed EEG in a first frequency band classifies whether the participant is a dementia patient by using a first supervised learning method; and
a coherence module selecting the processed EEG from different corresponding encephalic regions for coherence analysis, using a second supervised learning method to classify the non-dementia participants into healthy controls (HC) and mild cognitive impairment (MCI).

2. The cognitive impairment detection system according to claim 1, wherein the first supervised learning method is support vector machine (SVM), K-nearest neighbors (KNN), decision tree, random forest, logistic regression, Extreme gradient boosting (XGBoost), neural networks, or Naive bayes classifier.

3. The cognitive impairment detection system according to claim 1, wherein the second supervised learning method is support vector machine (SVM), K-nearest neighbors (KNN), decision tree, random forest, logistic regression, Extreme gradient boosting (XGBoost), neural networks, or Naive bayes classifier.

4. The cognitive impairment detection system according to claim 1, wherein the first frequency band is 0.5 to 20 Hz.

5. The cognitive impairment detection system according to claim 1, wherein the PSD module selected the processed EEG from the corresponding encephalic region of left frontopolar (Fp1), left frontal (F3), right frontal (F4), right posterior temporal (T6), midline parietal (Pz), or central midline (Cz).

6. The cognitive impairment detection system according to claim 1, wherein the coherence module selecting the processed EEG between right frontopolar (Fp2) and left temporal (T3), midline frontal (Fz) and left central (C3), right frontal (F4) and left central (C3) for coherence analysis.

7. A cognitive impairment detection method, comprising:
collecting an original electroencephalogram (original EEG) from a corresponding encephalic regions of a participant;
processing the original EEG to form a processed electroencephalogram (processed EEG); and
performing feature extraction step to classify the participant, wherein the feature extraction step comprising:
extracting a power in the first frequency band based on the processed EEG and using a first supervised learning method to determine whether a participant is a dementia patient; and
performing a second supervised learning method based on the coherence analysis in the processed EEG to determine whether the non-dementia participants is a healthy controls (HC) or a mild cognitive impairment (MCI).

8. The cognitive impairment detection method according to claim 7, wherein the first frequency band is 0.5 to 20 Hz.

9. The cognitive impairment detection method according to claim 7, wherein the first supervised learning method is support vector machine (SVM), K-nearest neighbors (KNN), decision tree, random forest, logistic regression, Extreme gradient boosting (XGBoost), neural networks, or Naive bayes classifier.

10. The cognitive impairment detection method according to claim 7, wherein the second supervised learning method is support vector machine (SVM), K-nearest neighbors (KNN), decision tree, random forest, logistic regression, Extreme gradient boosting (XGBoost), neural networks, or Naive bayes classifier.
